(19) United States
(12) Patent Application Publication
(10) Pub. No.: US 2010/0191232 A1
(43) Pub. Date: Jul. 29, 2010

(54) CATHETERS AND METHODS FOR PERFORMING ELECTROPHYSIOLOGICAL INTERVENTIONS

(75) Inventor: Serge Boveda, Toulouse (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Boveda Marco Medical LLC, Fremont, CA (US)

(21) Appl. No.: 12/694,852

(22) Filed: Jan. 27, 2010

Related U.S. Application Data
(60) Provisional application No. 61/147,721, filed on Jan. 27, 2009.

(51) Int. Cl. A61B 18/14; A61B 18/18

(52) U.S. Cl. 606/33; 606/41

(57) ABSTRACT

An electrophysiological ablation catheter comprises a flexible shaft having a circumferential tissue ablation structure and a linear tissue ablation structure at its distal end. By positioning the circumferential tissue ablation structure in a pulmonary vein ostium and the linear tissue ablation structure extending radially from the pulmonary vein ostium toward a second pulmonary vein ostium, lesions around and between the ostium may be formed with a single catheter placement. Repositioning the catheter allows the remaining pulmonary vein ostia and tissue therebetween to be ablated in order to treat atrial fibrillation.

### CROSS-REFERENCES TO RELATED APPLICATIONS

The present application claims the benefit of U.S. Provisional Patent Application No. 61/147,721 (Attorney Docket No. 027848-000100US), filed Jan. 27, 2009, the entire contents of which are incorporated herein by reference.

### BACKGROUND OF THE INVENTION

1. Field of the Invention

The present invention relates generally to medical devices and methods. More particularly, the present invention is directed at cardiac catheters having circumferential and/or linear ablation structures which allow treatment around and between pulmonary arteries of a patient's heart.

Atrial fibrillation is a supraventricular tachyarrhythmia characterized by uncoordinated atrial activation with associated deterioration of atrial mechanical function. During atrial fibrillation, normal electrical impulses that are generated by the sinus node are overwhelmed by disorganized electrical impulses that originate throughout the atria and pulmonary veins. These impulses upon transmission to the atrioventricular node can produce irregular and rapid heartbeats. Such irregular heartbeat may result in the pooling of blood within the atria, thus increasing the risk of thromboembolism and ultimately stroke.

Various classification systems have been proposed for atrial fibrillation. There is a general clinical consensus that recognizes three patterns of atrial fibrillation: paroxysmal (lasting less than 7 days and self-terminating), persistent (lasting more than 7 days and requiring electrical or pharmacological cardioversion), and permanent (cardioversion failed or not attempted).

The onset and maintenance of a tachyarrhythmia require both an initiating event (trigger) and an anatomical structure (or substrate). Two mechanisms are proposed for atrial fibrillation. The first is a “focal” mechanism involving automaticity or multiple reentrants wavelets often associated with paroxysmal atrial fibrillation, and the second involves the presence of macroreentrant loops and/or multiple reentrants wavelets meandering throughout the atria seeking nonrefractory tissue often associated with persistent atrial fibrillation. The focal source hypothesis is consistent with a dominant role for the left atrium in human atrial fibrillation. Atrial fibrillation can be initiated by ectopic beats originating from the pulmonary veins or elsewhere. This hypothesis provides the rationale for ablation procedures that isolate pulmonary vein ostia responsible for such ectopic beats. The second theory involving the presence of macroreentrant loops provides the rationale for ablation procedures that compartmentalize the left atrium area available for conduction and therefore reentry.

Management of atrial fibrillation involves one or more of rate control, thromboembolism prevention, and correction of the rhythm disturbance. Depending on the nature of the disease, one or several treatment options may be followed, such as lifestyle change, pharmacological therapy, electrical cardioversion and catheter ablation. The latter has emerged as a realistic treatment strategy to target pulmonary venous triggers that initiate atrial fibrillation. Ablation is at this time the only curative treatment for atrial fibrillation.

Paroxysmal atrial fibrillation, especially of short duration, is frequently a purely trigger-dependent phenomenon, whereas persistent and permanent atrial fibrillation are generally mechanistically complex, implicating a more diffuse abnormality of the atrial substrate. This heterogeneity of substrate may explain why no single predetermined ablation schema is effective for all patients across the entire spectrum of atrial fibrillation.

At present there are at least three principal techniques for catheter ablation to treat atrial fibrillation. Pulmonary vein isolation is often sufficient to treat paroxysmal atrial fibrillation. Linear ablation is usually directed at left atrium electrophysiological targets such as fractionated potentials or “rotors”. Linear and/or fractionated potentials ablation can be combined with pulmonary vein isolation to treat persistent atrial fibrillation.

Currently, pulmonary vein isolation is usually performed using circular ablation catheters, while linear ablation and ablation of left atrium electrophysiological targets is usually performed using single point tip-electrode ablation catheters. Clinical use of such single point techniques presents significant challenges in creating continuous lines without gaps inside the beating heart. For this reason, the success rate of atrial fibrillation ablation is modest at the present time (less than 50% for persistent atrial fibrillation).

There is therefore a need for improved catheter ablation devices to treat atrial fibrillation, in particular ablation catheters that can be used for isolation of electrical potentials and targeting the compartmentalization of different areas within the atria of the heart.

2. Description of the Background Art

Cardiac ablation catheters are described in U.S. Pat. No. 7,429,261; US2002/0087151; US2005/0065512; US2005/0251132; and US2008/0281312.

### SUMMARY OF THE INVENTION

The present invention provides apparatus and methods for performing electrophysiological interventions, particularly cardiac ablations, in and around the pulmonary veins for the treatment of atrial fibrillation. Catheters according to the present invention will be configured to engage a first venous ostium while having a linear tissue ablation structure which may be deployed from the engaged venous ostium toward a second venous ostium to produce a continuous linear lesion therebetween. In certain preferred embodiments, the catheters will also include a circumferential tissue ablation structure which is adapted to engage the first venous ostium to produce a continuous circular lesion thereabout so that both the circumferential lesion and the linear lesion may be formed with a single catheter placement. Thus, by sequentially repositioning the catheter from the first venous ostium, to the second venous ostium, to a third venous ostium, and finally to a fourth venous ostium, the regions in and a around the venous ostia may be completely isolated in a single procedure using four sequential placements of the same catheter.

Thus, in a first aspect, the present invention provides an electrophysiological ablation catheter comprising an elongate flexible catheter shaft having a distal extremity and a proximal extremity. A connector hub is provided at the proximal extremity, and the distal extremity is adapted for insertion into an atrium of a patient's heart, typically by a transseptal access route. A circumferential tissue ablation structure is deployably coupled on the distal extremity of the shaft, and a linear tissue ablation structure is deployably coupled to the distal extremity of the shaft, where the linear ablation structure intersects the circumferential electrode structure at least one point. An energy conduction structure is provided between the tissue ablation structures and the connector hub to allow connection of the catheter to conventional radio frequency or other ablation energy sources. The circumferential tissue ablation structure is adapted to engage a first venous ostium while the linear tissue ablation structure extends radially from the first ostium toward a second venous ostium to produce a substantially continuous circular lesion in and around said first ostium and a substantially continuous linear lesion between said first ostium and second ostium, usually intersecting the continuous circular lesion.

The electrophysiological ablation catheters of the present invention will typically have an array of electrodes spaced apart on at least one of the circumferential and linear tissue ablation structures, typically on both, so that they may deliver radio frequency ablation energy from a conventional radio frequency power supply. Alternatively, the ablation structures of the present invention may be adapted to deliver other energy modes, including ultrasound, microwave, thermal energy (including cryogenic energy), and the like. When connecting to individual electrodes in electrode arrays on the ablation structures, the energy conduction structure will usually comprise a plurality of individual electrical conductors which connect to individual ones of said electrodes in said electrode arrays so that the electrodes can be selectively activated individually or simultaneously in discreet groups to produce continuous lesions of varying diameter or length.

The circumferential tissue ablation structure will typically have a diameter in the range from 20 mm to 30 mm to allow for the creation of both small and large diameter lesions in or around the venous ostium. The linear tissue ablation structures will typically have a length from 20 mm to 100 mm suitable for the creation of both short and long lesions. Optionally, the catheter may include lumens or passages that allow flow of an aqueous solution through or in between individual electrodes or other portions of the ablation structures. Optionally, the catheter shaft may be steerable, for example including a mechanism for deflecting and steering the distal extremity of the shaft. Steering is advantageous since it allows the user to move the circumferential support structure in or around any of the pulmonary vein ostia within the left atrium or other anatomical structures of the heart. Further optionally, the catheters may comprise a torquing mechanism for rotating a distal portion of the distal shaft about a longitudinal axis in order to radially position the linear ablation structure relative to the access.

The catheters of the present invention may further comprise a deployable, self-centering anchor member to promote catheter retention within the venous ostia or elsewhere to position the distal extremity of the shaft axially and radially relative to the ostium. For example, the anchor may comprise a balloon, typically a spherical balloon having a diameter in the range from 10 mm to 30 mm when inflated. Optionally, the balloon may be present on a support structure that is longitudinally positionable at or from the distal extremity of the shaft, either independently or coupled with the deployment mechanism for the circumferential and linear electrode structures. Alternatively, the anchoring member may comprise an expandable basket or further alternatively, a malecot having a plurality of wings which are deployed to promote catheter retention.

The circumferential and/or linear electrode ablation structures will typically comprise a plurality of discreet electrodes, as described previously, or may alternatively comprise a unitary conductor. Further optionally, the unitary conductor may have an insulating sheath positionable over its length in order to selectively expose the contact surface of the electrode to tissue. Still further alternative ablation structures may comprise bridged electrodes which form a single conductive path. Usually, the bridges will be recessed or insulated so as to not contact tissue upon deployment of the electrode structure.

The present invention further comprises systems including an electrophysiological ablation catheter as described above in combination with a power supply which may be coupled to the catheter, where the power supply is capable of transferring a tissue ablative energy between the power supply and the tissue ablation structures of the catheter. For example, the power supply may be configured to deliver electromagnetic energy, acoustic energy, thermal energy (including low temperature thermal energy), and mechanical energy. Typically, the power supply will deliver radio frequency energy to electrode(s) of the ablation structure(s). Optionally, the power supply may be further configured to sense electrical activity of the endocardial tissue, optionally through the electrodes of the ablation structures. Still further optionally, the power supply may be adapted to sense and display tissue temperature, tissue impedance, or other tissue parameters.

In a further aspect of the present invention, methods for treating atrial fibrillation comprise advancing an electrophysiological ablation catheter to an atrium of a patient's heart. A circumferential ablation structure is deployed from the catheter to engage a first venous ostium. A linear ablation structure is also deployed from the catheter so that the linear ablation structure extends radially from the first venous ostium toward a second venous ostium. The circumferential and linear ablation structures are energized to produce a substantially continuous circular lesion surrounding the first venous ostium and a substantially continuous linear lesion on the interior surface of the heart intersecting the circular lesion and extending from the first venous ostium towards the second venous ostium. Optionally, electrical activity in the heart may be sensed through one or more set of ablation structures to determine the presence of aberrant conductive pathways and to map the inner surfaces of the heart, either before or after ablation treatment. Usually, the electrophysiological ablation catheter is positioned using an anchoring member coupled to its distal end within the first venous ostium to radially and longitudinally position the catheter prior to deploying the circumferential and/or linear ablation structures.

After the first venous ostium has been treated and the first linear lesion formed toward the second venous ostium, the second venous ostium may be treated by deploying the circumferential ablation structure on the catheter to engage the second venous ostium, typically by repositioning a distal end of the ablation catheter within the atrium. The linear ablation structure is then deployed to extend from the second venous ostium toward a third pulmonary venous ostium, and the circumferential and linear ablation structures energized as described previously to create continuous circumferential and linear lesions, where the linear lesion extends toward a third pulmonary venous ostium.

It will be appreciate that this step-wise treatment may be continued as needed to complete isolation of the pulmonary veins and tissue structures that are between. It will be further appreciated that the number of pulmonary veins treated will usually be four, but in some patients may be as few as three or as many as six. The catheter structure and methods of the present invention are ideally suited for treating any number of pulmonary veins.

In another aspect of the present invention, an electrophysiological catheter may comprise an elongated flexible shaft having a distal extremity and a proximal extremity. A connector hub is provided on the proximal extremity and the distal extremity is adapted for insertion into a atrium of a patient's heart. A deployable self-centering anchor member is provided at the distal end of the distal end extremity of the shaft and is adapted to promote catheter retention within a first venous ostium in order to position the distal extremity axially and radially relative to the first ostium. At least a linear tissue ablation structure will be deployably coupled to the distal extremity of the shaft, where the linear ablation structure deploys radially outwardly from the shaft. Optionally, a circumferential tissue ablation structure will also be deployably coupled to the distal extremity, typically where the linear tissue ablation structure and circumferential tissue ablation structure intersect as with the previously described embodiments. The electrophysiological ablation catheter will further include an energy conduction structure extending between the tissue ablation structures and the connector hub, and the linear tissue ablation structure will deploy radially from the first ostium toward a second venous ostium to produce a continuous linear lesion therebetween when the anchor member is deployed in the first venous ostium. When the circumferential tissue ablation structure is present, it will be adapted to engage the pulmonary vein ostium while the linear electrode structure extends radially toward the other pulmonary vein. The details of the linear ablation structure and the circumferential tissue ablation structure will be the same as those described above with respect to the earlier embodiments of the present invention, and the deployable self-centering anchor member may also have any of the specific structures discussed above with respect to the earlier embodiments.

### BRIEF DESCRIPTION OF THE DRAWINGS

FIG. 1 illustrates a system comprising an electrophysiological ablation catheter constructed in accordance with the principals of the present invention connected to other system components useful for performing cardiac ablation procedures.

FIGS. 2 and 3 illustrate first embodiment of an ablation catheter constructed in accordance with the principals of the present invention.

FIGS. 4-6 illustrate alternative constructions for the carrier assembly portion of the ablation catheter of FIGS. 2 and 3.

FIG. 7 illustrates a catheter shaft deflection mechanism useful in the catheters of the present invention.

FIGS. 8-10 illustrate deployment of the ablation structures of the catheters of the present invention.

FIGS. 11 and 12 illustrate alternative anchor mechanisms useful with the catheters and methods of the present invention.

FIGS. 13-23 illustrate use of the ablation catheter of FIGS. 2-6 for sequentially ablating the pulmonary ostia and tissue between adjacent pulmonary ostia and a patient in accordance with the methods of the present invention.

### DETAILED DESCRIPTION OF THE INVENTION

While the present invention will be described with specific reference to a limited number of embodiments, this description is illustrative and should not be construed as limiting to the invention. Modifications to this invention can be made to the preferred embodiments by those skilled in the art without departing from the true spirit of the invention as defined by the claims. It will be noted here that for a better understanding, like components are designated by like reference numerals throughout the various figures.

The present invention provides catheters and methods for treating atrial fibrillation. The catheters comprise one or more electrode array(s) disposed at the distal end of a catheter body on either or both a circumferential structure and linear structure. The arrays have specific geometric configurations that allow them to engage and/or ablate specific atrial tissue in order to electrically isolate or segment structures within the atrium. The catheters transseptally access the left atrium of the heart of a patient through a puncture in the atrial septum and ablate tissue surrounding the four pulmonary veins (the two superior and the two inferior) in order to isolate them from the rest of the left atrium, and create at least two continuous lines of ablation, one at the roof of the left atrium and one at the mitral isthmus in order to obtain a left atrial segmentation.

The catheters may optionally have a deployable self-centering anchor member to promote catheter retention or stability at the distal end of the distal extremity of the shaft, wherein said anchor member is deployable within a venous ostium to position the distal extremity axially and radially relative to the ostium. Examples of self-centering anchor members include but are not limited to inflatable balloons, self-expandable baskets, and multi-winged malecots.

The use of electrophysiological catheters with deployable self-centering anchor members as described above, will significantly improve catheter ablation procedures used to treat atrial fibrillation. The self-centering anchor member can be directed over a guidewire into a target anatomical structure, for example, one of the pulmonary veins within the left atrium. Once in place, the self-centering anchor member can be deployed and secured within the pulmonary vein. In doing so, the circumferential structure of the catheter can be securely and accurately positioned and held in place at the ostium of the pulmonary vein. The linear structure can also be positioned relative to the circumferential structure and apposed to an atrial wall. The self centering anchor can provide stability to optimize apposition of the circular or linear electrode arrays. Delivery of ablation energy from a plurality of electrodes on the circumferential and/or linear structures can be used to create a fully continuous and circumferential lesion in or around the ostium of the pulmonary vein and a linear lesion extending from and connected to said circumferential lesion typically with a single delivery of energy.

Using the catheters and methods of the invention, the isolation of the pulmonary veins as well as the linear lesions described above may be accomplished using a single catheter in one intervention. Being equipped with both a circular or spiral electrode array configured to encircle the pulmonary vein ostia, as well as a linear electrode array suitable for linear ablation of the atrial wall, the catheters of the invention allow more precise and stabilized ablation procedures while reducing the time, cost, and potential morbidity associated with multiple-catheter procedures.

Currently available catheters are not well suited at performing continuous linear lesions in the left atrium. The lines of ablation in the left atrium are currently performed mainly through single electrode (4 mm) standard catheters, guided or not guided by 3-D mapping systems. The lines are made point by point, with all its faults and difficulties due to lack of stability and poor contact with the wall of the left atrium. The procedures are complicated, imperfect, and typically require a long total duration and also long fluoroscopy time. Such shortcomings lead to a low success rate and an increased risk of complications. The ability to now create both circumferential and linear continuous lesions with a single electrophysiological ablation catheter will greatly reduce procedure times and risks to the patient.

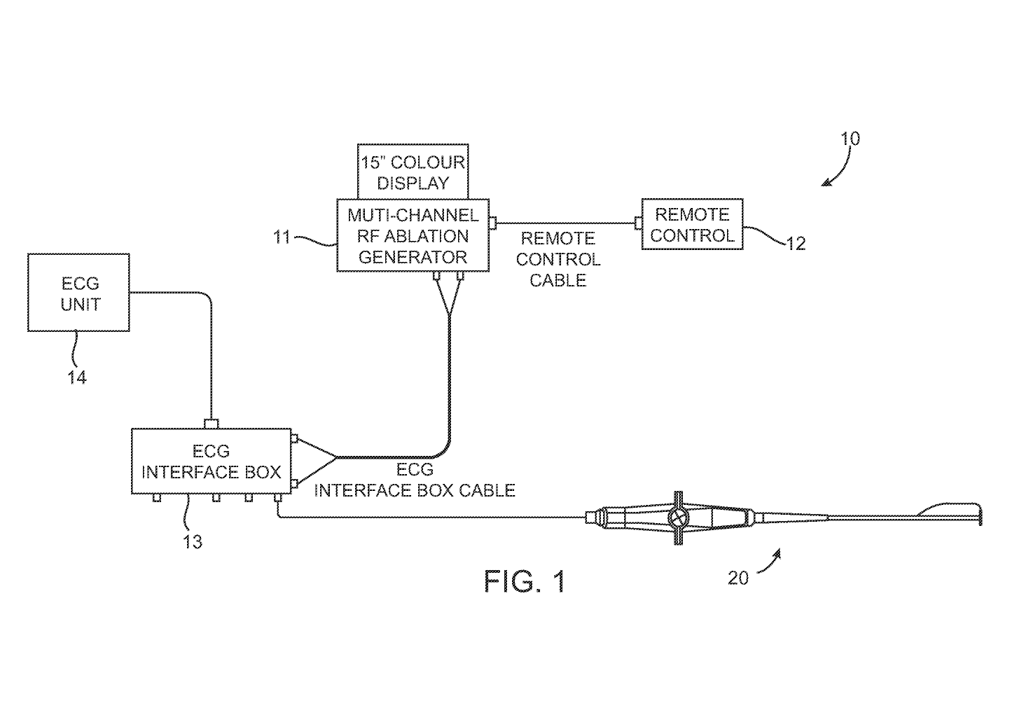

Referring to FIG. 1, an endovascular ablation therapy system 10, is provided for systematically treating atrial fibrillation. “Atrial Fibrillation” as used herein includes paroxysmal (lasting less than 7 days and self-terminating), persistent (lasting more than 7 days and requiring electrical or pharmacological cardioversion), and permanent (cardioversion failed or not attempted) atrial fibrillation. The ablation therapy system 10 includes a power supply, such as multi-channel RF ablation generator 11 typically including a remote control 12, an ECG interface box 13, an ECG unit 14, and an electrophysiological ablation catheter 20.

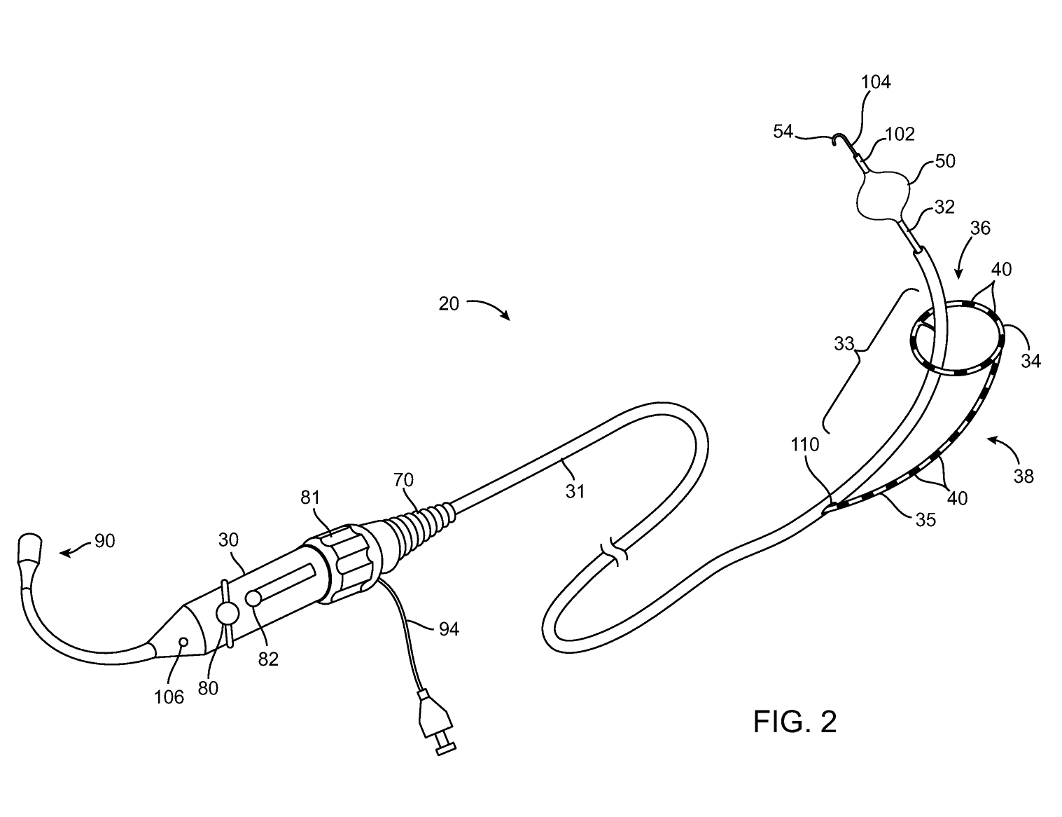

Referring to FIG. 2, an ablation catheter 20 includes a handle 30, an elongated, flexible outer tube 31 that defines a lumen that slidably receives a control shaft 32 therethrough, and a distal carrier assembly 33 on the distal extremity of the catheter adapted for insertion into and positioning within an atrium of a patient's heart. The distal carrier assembly 33 includes a circumferential structure 34 (the circumferential structure can also be referred to as a “spiral” or “circle” or “loop”) and a linear structure 35 deployably coupled to the distal end of the catheter. The circumferential structure 34 and linear structure 35 may optionally each include an array of electrodes 40. The circumferential and linear structures and electrode arrays are resiliently biased and have specific geometric configurations that generally allow them to contact and ablate specific atrial tissue or specific areas within the atrium, such as the ostium of a pulmonary vein and the region between adjacent pulmonary vein os. Each electrode array is selectively movable from a stored or delivery configuration for transport and delivery (such as a radially constrained configuration) to a deployed or expanded configuration for tissue ablation.

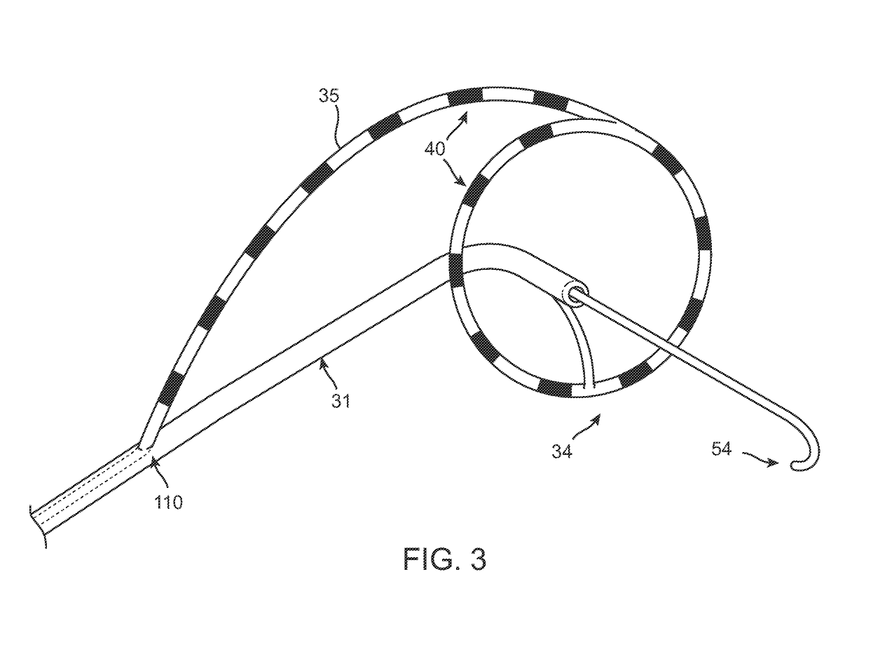

Referring to FIGS. 2 and 3 the catheter 20 includes the handle portion 30, the elongated, flexible outer tube 31 that defines a lumen that slidably receives a control shaft 32 therethrough, and the distal carrier assembly 33 on the distal extremity of the catheter adapted for insertion into an atrium of a patient's heart. The outer catheter tube 31 is formed and dimensioned to provide sufficient column and torsional strength to support standard interventional procedures such as those which access the vasculature from a femoral vein and further access the patient's heart. As shown in FIG. 2, a capture device 70 is friction fit over the distal end portion of the handle portion 30. This device 70 is configured to be detached therefrom and slide in a distal direction over the catheter tube 31 until the circumferential and linear structures 34, 35 are received therein, in the stored or confined configuration. As will be described, the capture device 70 is applied over the structures 34, 35 for constraint and protection thereof during delivery through a hemostasis valve of a trans-septal sheath or a vascular introducer. In this manner, the circumferential and linear structures 34, 35 with electrodes arrays may be introduced safely (e.g. without damage) into the patient's vasculature (e.g., a femoral vein). After introduction through the hemostasis valve, capture device 70 is moved proximally over catheter tube 31 and reattached to the distal end portion of the handle portion 30 to function as a strain relief.

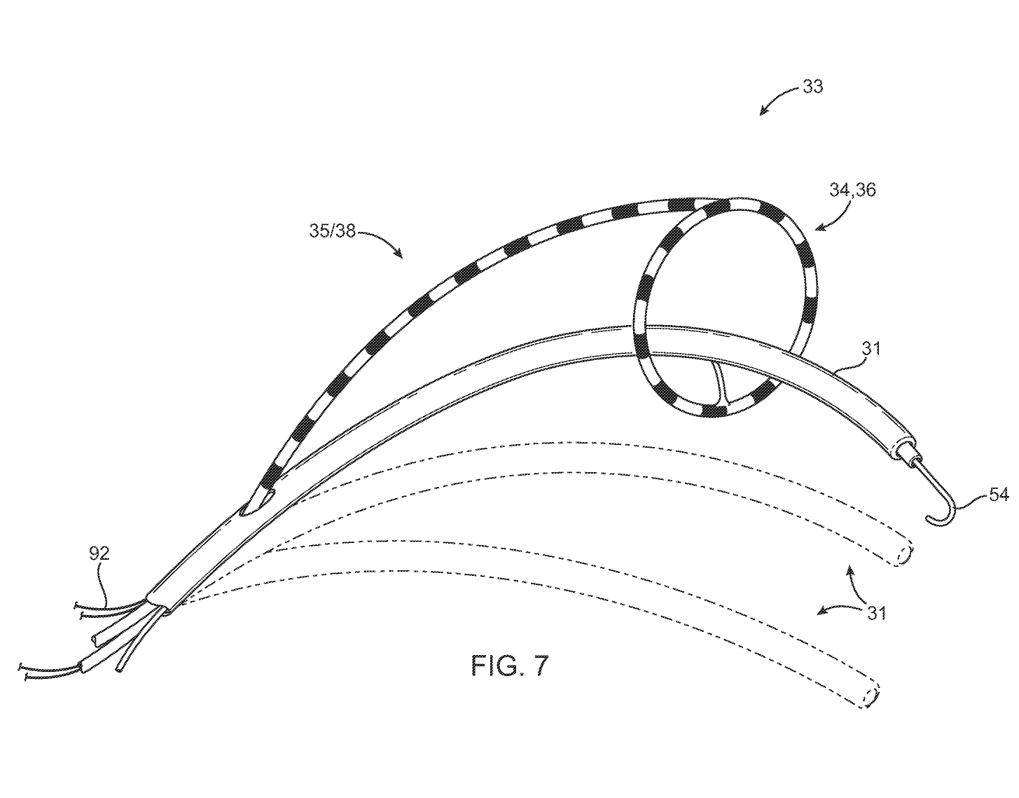

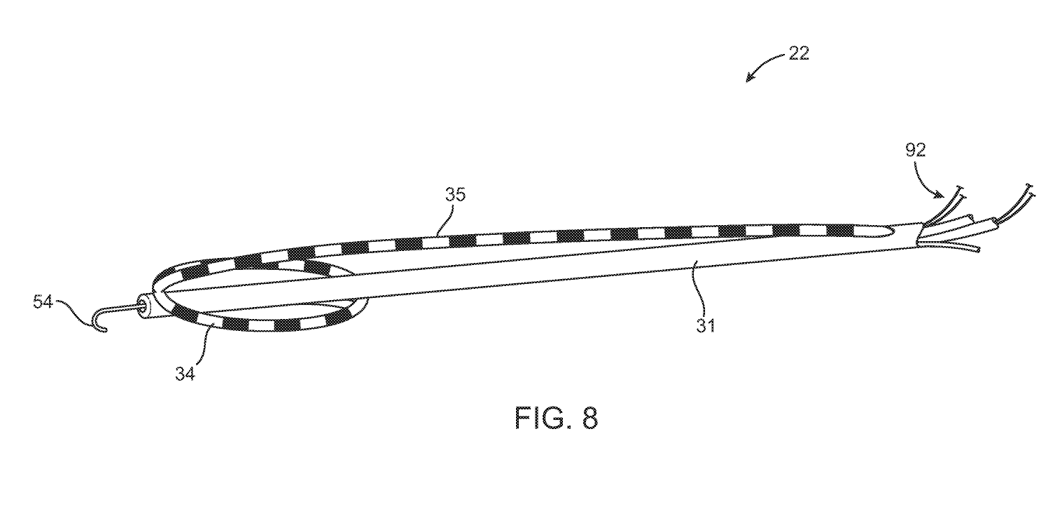

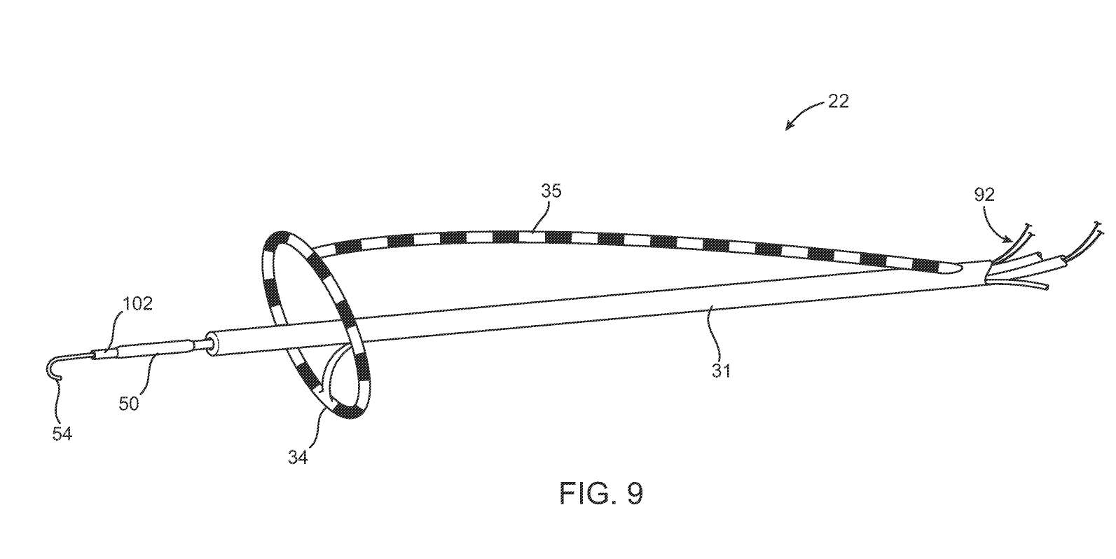

The handle portion 30 includes a deployment control knob 80, a steering control knob 81, a push/pull control knob 82 and an electrical connector or plug 90. One or more internal push/pull wires 92 (FIG. 7) are provided, having one end affixed to the outer catheter tube and an opposite end coupled to the steering control knob 81 to enable steering thereof. The elongated flexible outer catheter tube 31 is mounted on the end of the handle portion 30, and the circumferential 34 structure and linear structure 35 are mounted at the distal end of the catheter tube 31. Optionally a self-centering anchor member 50 (for example, a balloon) may be provided at the distal end of the control shaft 32. The distal end of the device is adapted to be deformable such that pressing the distal loop (i.e. the circumferential structure 34) into a pulmonary vein ostia 48 or left atrial wall 56 will cause electrode array 36 on the circumferential structure 34 to conform to an inner surface of the atrium. Similarly electrode array 38 on the linear structure 35 will conform against the inner wall of the atrium, and all of electrodes 40 will make sufficient contact with tissue to deliver RF energy and/or sense electrical potentials. Usually, in order to avoid clot formation, an infusion link 94 will be provided to allow heparinized infusions inside the inner lumen of the catheter. The linear structure 35 includes a flexible support arm having a distal end coupled to the circumferential structure 34 and proximal end coupled to the central control shaft 32. The circumferential support structure 34 is coupled at one point to the linear support structure 35 and at another end to the flexible outer tube 31. By sliding and rotating the control shaft 32, such as rotating control knob 80 on the handle portion 30, the carrier assembly 33 can be manipulated to control the geometry of both the circumferential and linear support structures. For example, the control shaft 32 can be retracted to transition the linear structure 35 from a near linear configuration (as shown in FIG. 8) to an arcuate configuration (as shown in FIG. 9). Advancement and/or retraction of the control shaft 32 adjusts the geometry of the linear structure, such as increasing or decreasing the curvature, i.e. the degree to which the linear structure can be made arcuate. As the control shaft 32 is retracted further, the circumferential structure 34 is fully deployed from its stored position.

The diameter of the circumferential structure 34 will typically be in a range from about 15 mm to a of about 35 mm, and the preferred range of usable lengths of the linear structure 35 is typically about 20 mm to a maximum length of about 80 mm, to accommodate the varied anatomical contours neighboring pulmonary vein ostia (including non-circular ostia or common trunks) and inner atrial wall anatomies respectively. A slide knob 82 is provided on the handle portion 31 to advance and retract the control shaft to deploy the linear structure 35 in order to obtain a better contact against the left atrial wall.

The catheters will be used with the RF Generator 11 (FIG. 1) configured to deliver bipolar and/or monopolar ablative energy to the catheters, and an ECG interface 13 coupling an ECG monitoring unit 14 to the RF generator 11. In one specific embodiment, the ECG interface unit 13 is configured to isolate the RF generator 11 from the ECG monitoring unit 14. In particular, the circuitry electrically isolates potentially damaging signals generated by the RF generator 11 from the ECG unit 14, as well as shielding the unit from other electrical noise (FIG. 1).

In some embodiments, the catheter electrode arrays may be adapted to measure the temperature of atrial tissue adjacent the electrodes 40 (with, for example, a thermocouple). The generator 11 monitors the measured temperature and delivers energy to the electrode based on the measured temperature. A temperature feedback loop is thereby generated between the electrode and the generator. The generator can be adapted to independently monitor the temperature of atrial tissue measured by more than one thermocouple in the arrays, and the RF generator can then generate and deliver RF energy to individual electrodes based on the independently monitored temperatures. When operating in bipolar mode, the generator can selectively limit the amount of energy being delivered to an electrode pair if the electrode is measuring the adjacent atrial tissue to be higher than the temperature of the tissue measured by the second electrode in the pair of electrodes.

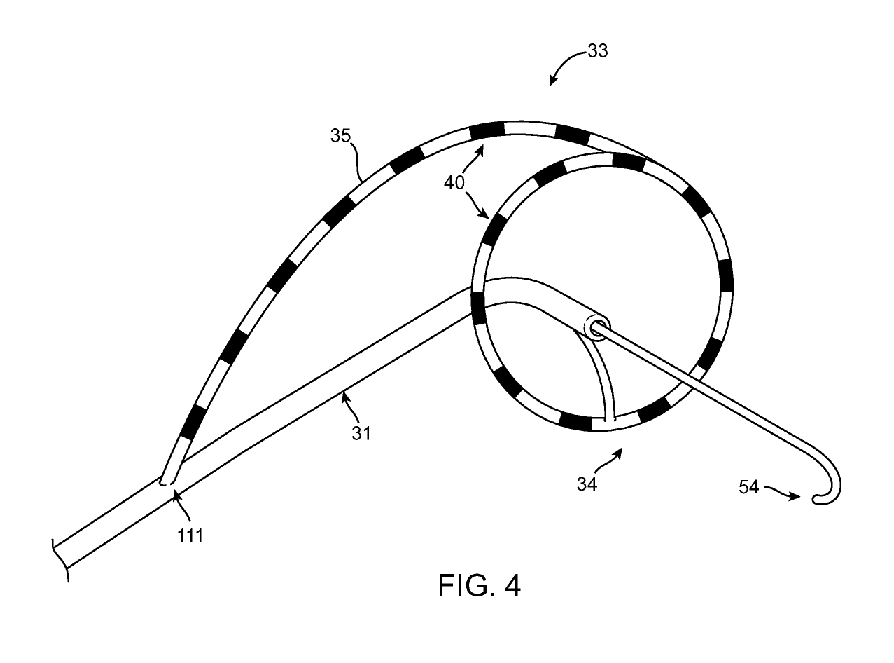

Several variations of the distal carrier assembly 33 of the catheter 20 illustrated in FIGS. 2 and 3 are shown in FIGS. 4-6. In FIG. 4, the distal carrier assembly 33 comprises a linear support structure 35 which is fixedly attached at location 111 to the outer tube 31. In this case, deployment of both the linear support structure 35 and the circumferential support structure 34 may be accomplished using an outer sheath (not shown) which is advanced over the support structures to collapse said structures into a small diameter low profile configuration for advancement to the atrium. Once in place, the structures may be deployed by retracting the sheath to permit the structures to self-expand into the geometry illustrated in FIG. 4.

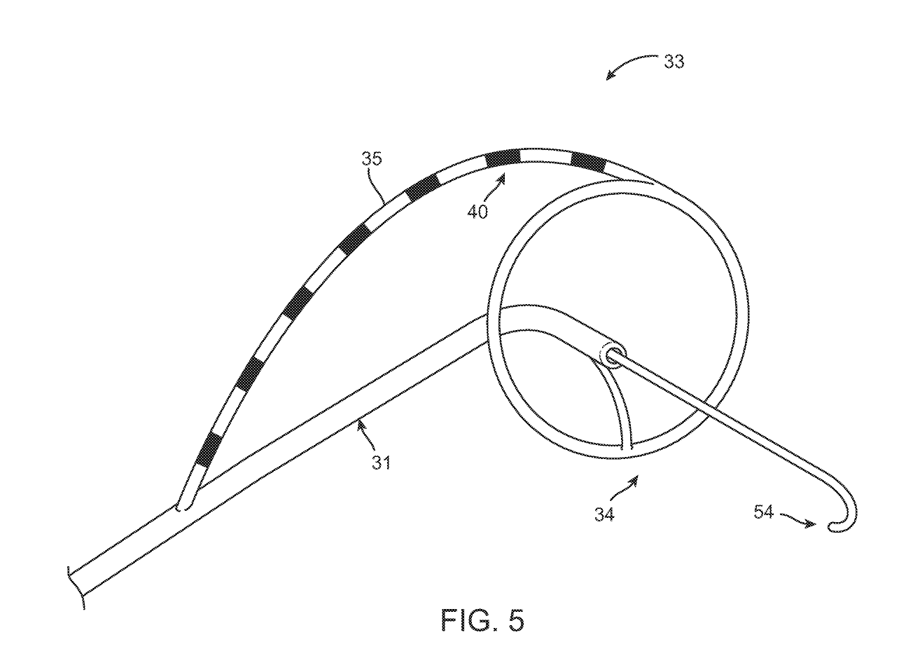

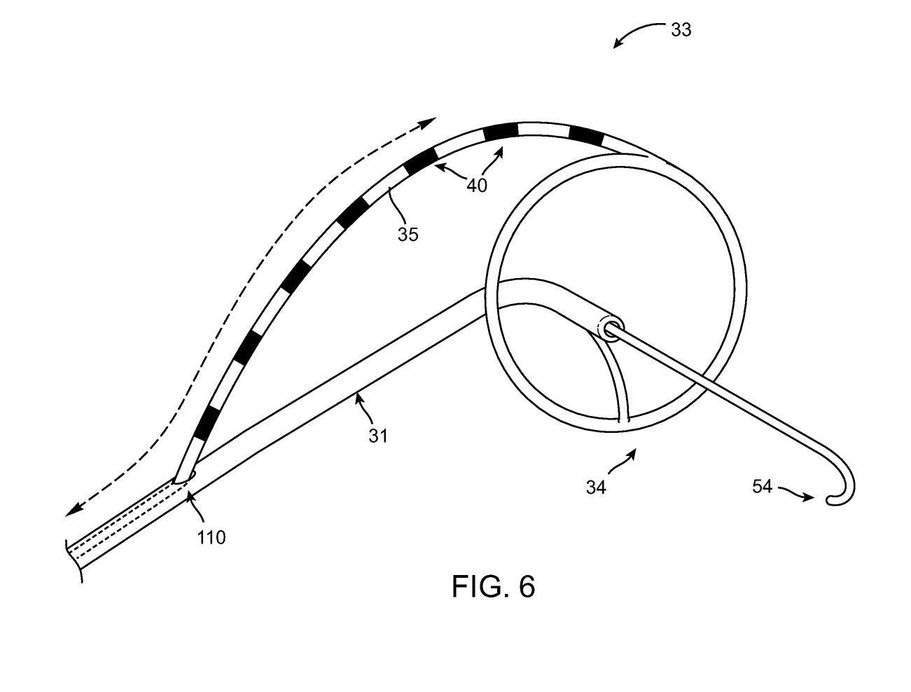

FIG. 5 illustrates a carrier assembly 33 which is similar to that shown in FIG. 4, except that the circumferential support structure 34 does not include an ablation structure, i.e., it is free from electrodes or other energy delivery components. Catheters having the carrier assembly of FIG. 5 will be useful for making linear ablations while the catheter is stabilized within the ostia using the circumferential support structure 34. The carrier assembly 33 in FIG. 6 is similar to that of FIGS. 2 and 3, except that the circumferential support structure is free from electrodes or other energy delivery components. As with the embodiment of FIG. 5, catheters having the carrier assembly 33 of FIG. 6 will be useful for forming linear lesions when it is not necessary to form circumferential lesions.

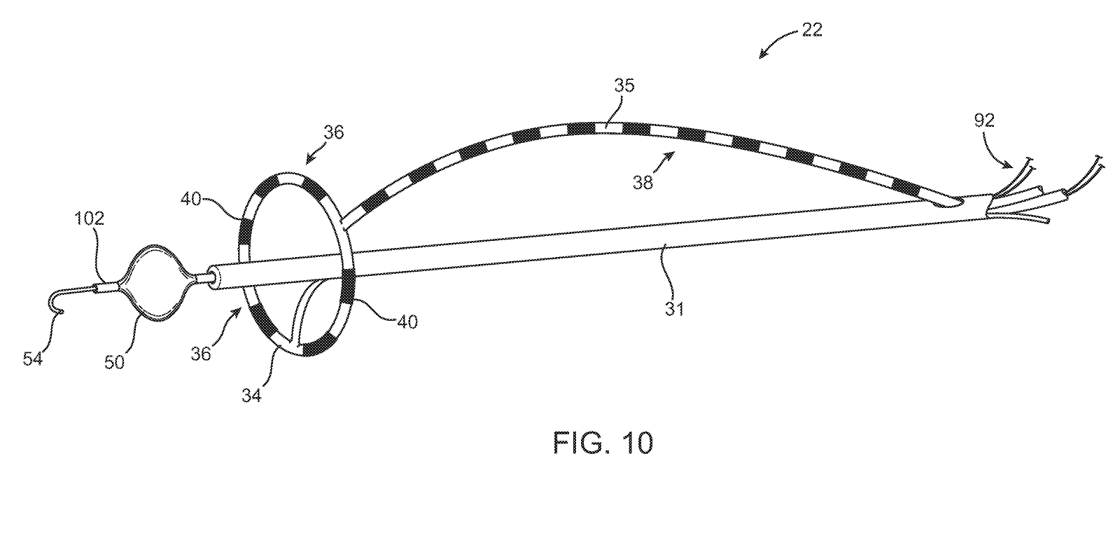

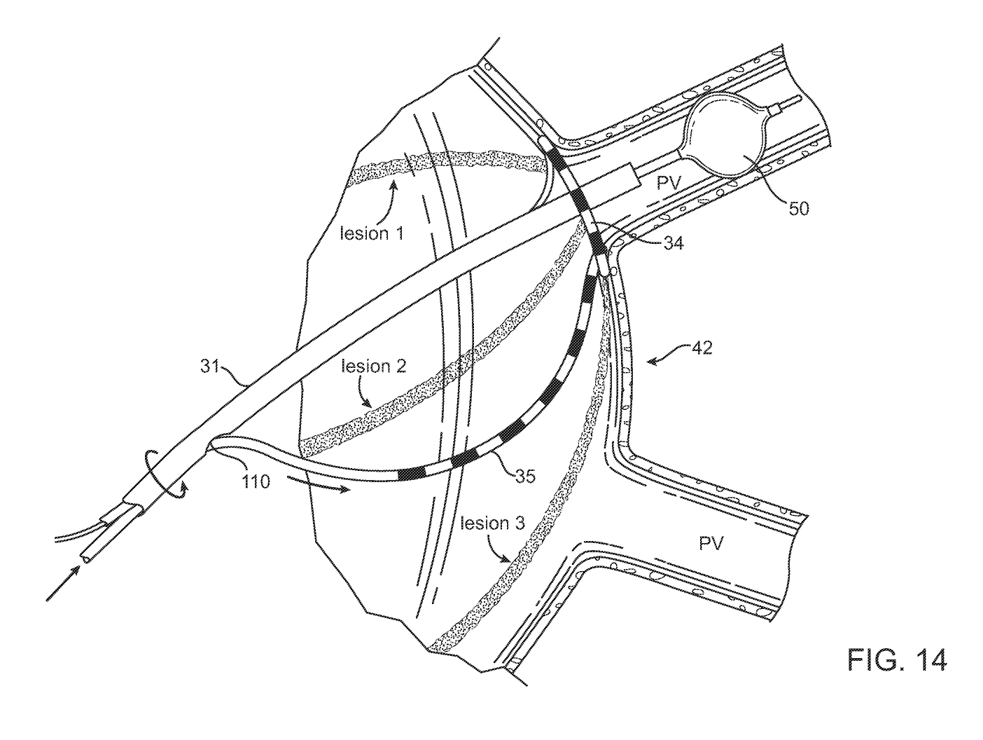

FIGS. 8-10 show three stages of deployment of the electrophysiological ablation catheter. For intraluminal transport through a vessel, both the circumferential and linear structures 34, 35 are parallel, in a radially constrained configuration, FIG. 8. The capture device 70 may be applied over these structures while in the confined configuration for constraint and protection during delivery through a hemostasis valve of a transseptal sheath or a vascular introducer. FIG. 9 is illustrative of the catheter in a stage intermediate between its stored configuration and the fully deployed configuration, FIG. 10. In the deployed configuration (within the left atrium 42), the circumferential structure 34 is perpendicular to the outer tube 31 and the linear structure 35 bows outwardly from the tube 31 (see FIG. 14).

The electrodes 40 of the circumferential 34 and linear structure 35 may be mounted to detect electrical signals between any pair of electrodes (bi-pole) for mapping of electrical activity, and/or for performing other functions such as pacing of the heart. Moreover, these electrodes deliver ablation energy across an electrode pair or from independent electrodes when delivering unipolar energy, using one of the pre-programmed settings. Preferably, four to twenty-two electrodes 40, and more preferably eight to sixteen electrodes, are positioned along the linear array 36 and circumferential array 38 with constant or varying spacing. Each electrode 40 can have an integral thermocouple (not shown) located on or near the tissue side of the electrode to monitor the temperature at each ablation site before and during ablation. The electrodes 40 are preferably made of platinum, and are typically about 3 mm long and separated by about 1 mm to about 4 mm. The electrodes 40, can be composed of any material and be of any shape and have any geometric configuration so long as they can create continuous ablation lesions upon delivery of energy. The electrodes can optionally include passages that allow flow of an aqueous solution out of an inner cavity of at least one electrode so as to provide irrigation during the ablation procedure.

Each of the electrodes 40 is attached via connecting wires and one or more connectors, such as connector plug 90, to an RF ablation generator (FIG. 1). This RF ablation generator is also attached to a patch electrode, such as a conductive pad attached to the back of the patient, to enable the delivery of unipolar ablation energy.

The circumferential or linear support structure 34, 35 of the catheter 20 can have a unitary electrode or conductor (not shown), instead of a plurality of electrodes. The unitary conductor functions in the same manner the electrodes 40 described above do. An insulative sheath is positioned over the surface so as to selectively limit exposure of the unitary conductor to the atrial tissue. Alternatively, the unitary conductor can comprise an array of electrodes connected via bridges to form a single conductive path. Such bridges will usually be recessed so as to not contact the tissue upon deployment of the electrode structure. Alternatively, the bridges can be insulated so as to not contact tissue upon deployment of the electrode structure.

While monopolar and bipolar RF ablation energy are the preferred forms of energy to be delivered by the electrodes of the ablation catheters, it will be appreciated that other forms of ablation energy that may be additionally or alternatively emitted from the electrodes or other ablation elements include electrical energy, magnetic energy, microwave energy, thermal energy (including heat and cryogenic energy) and combinations thereof. Moreover, other forms of ablation energy that may be applied that are emitted from the array include acoustic energy, sound energy, chemical energy, photonic energy, mechanical energy, physical energy, radiation energy and a combination thereof.

Referring again to FIG. 2, an atraumatic tip 102 at the very distal end of the carrier assembly 33 defines a through-hole 104 into a guidewire lumen extending proximally through the control shaft 32 and terminating at a guidewire exit 106 at the handle portion 30. This enables the carrier assembly 33 and flexible outer catheter tube 31 to be percutaneously advanced over a guidewire 54, such as a guidewire which has had its distal end inserted into a pulmonary vein of the patient. Additionally, distal markers (for example, platinum) are placed at the distal end of the catheter to aid with positioning of the catheter during the procedure.

To facilitate single or bi-directional steering and control of structures 34, 35 a single full length pull wire 92 (or double pull wires such as in the case with bi-directional steering, three or four pull wires may optionally be used to increase the degrees of freedom of movement) is secured to the distal portion of the end of the control shaft 32. The pull wires 92 run proximally to the steering control knob 81. Rotation of the knob 81 pulls the wire that in turn controls the plane in which the electrodes contact tissue, as shown in FIG. 7.

To allow a better contact of the linear electrode array 38 with the left atrial wall 56, a single full length pull wire 92 (or double pull wires) is secured to the proximal portion of the linear structure 35. The pull wires runs proximally to the steering control knob 82. Pushing/pulling of the knob 82 pushes/pulls the wire that in turn controls the linear structure 35 which extends slidably through a through-hole 110 in order to obtain an optimized contact between tissue and electrodes. This specific mechanism will usually only be available on the catheters of FIGS. 2, 3 and 6.

To commence a procedure, the distal portion of catheter 20 is advanced through the patient's vasculature, via the femoral vein. The distal portion is then advanced into the right atrium (RA), preferably through the inferior vena cava (IVC), via a lumen of the transseptal sheath. The outer catheter tube 31 is sized for this advancement through the patient's vasculature, such as where the inserted shaft diameter is approximately 9-12 Fr, the shaft working length is approximately 115 cm and the overall length is typically 158 cm. The catheter 20 is inserted over guidewire 54, through the lumen of the transseptal sheath, and into the left atrium 42.

In order to advance the carrier assembly 33 through the vasculature and into the left atrium 42, the catheter 20 is oriented in the substantially linear transport configuration (FIG. 8) by advancing control shaft 32 distally, such as by manipulating a deployment control (e.g., knob 80) on the handle portion 30 of the catheter. In turn the linear structure 35 is urged toward the linear configuration. In this linear orientation, the carrier assembly is maximally compact in a transverse dimension, and can be easily advanced through the transseptal sheath.

The capture device 70 may then be detached from the distal end portion of the handle portion 30, and slid in a distal direction over the catheter tube 31 all the way up to the electrode arrays 36, 38. While holding the capture device against the electrode arrays 36, 38, the deployment control knob 80 may be operated incrementally to advance the array 36 distally and assure that the tip of the electrode array 36 is distal to the capture to prevent kinking thereof. The electrode structures 34, 35 may then be captured in, or received within the capture device 70, in the stored or confined configuration.

The tip of the capture device 70 can then be inserted into a hemostasis valve or the like of the sheath until the capture device is seated against the inner surface of a hub of the sheath, enabling the structures 34, 35 to be safely transferred into the lumen of the sheath, already in the transport configuration. The structures 34, 35 and outer catheter tube 31 are advanced into the lumen of the transseptal sheath about five to eight inches. The capture device 70 may be detached or otherwise moved away from the hub, and reattached to the distal end of the handle portion 30, functioning as a strain relief.

Once the distal portion of the carrier assembly 33 is advanced along the guidewire 54 and past the distal end of the transseptal sheath, using conventional fluoroscopy techniques, it enters the Left Atrium 42. When it is determined that the carrier assembly is sufficiently past the sheath, using conventional fluoroscopy techniques, deployment of the carrier assembly may commence. In one particular embodiment, for instance, deployment of the circumferential structure 34 may begin when a particular electrode 40 along the linear structure 35, such as the third or fourth electrode, is advanced out of and past the end of the sheath.

To deploy the carrier assembly 33, as previously mentioned, the control shaft 32 is retracted relative to the distal end of the outer catheter tube 31, via manipulation of the handle control knob 80. Thus, while the longitudinal length of the carrier assembly is decreasing, the radial dimension of the deploying circumferential structure 34 is increasing. The carrier assembly 33, thus, can be further advanced into the left atrium 42 while simultaneously retracting the control shaft 32 to deploy the electrode arrays 36, 38 until it is fully beyond the end of the transseptal sheath.

The catheter can optionally have a self-centering anchor member 50, 51, 52, as described above, this member is inserted over the guidewire 54 into the pulmonary vein (first, the guidewire is inserted into the pulmonary vein, then, the anchor member is positioned into the pulmonary vein) and inflated or expanded to promote catheter retention at the ostium of the vein (an illustrative example is provided in FIG. 14). The anchoring member can be longitudinally adjusted by retraction of the control shaft 32 so as to position it at the target position within the pulmonary vein, once at the target position the member can be inflated or expanded. The ability to longitudinally position the anchoring member facilitates positioning of the circumferential structure 34 in or around the ostium of a pulmonary vein and stabilizes the catheter system within the atrium thus ensuring accurate lesions are formed to treat the aberrant conductive pathways throughout the tissue.

The self-centering anchor member can be a balloon, 50 (FIGS. 2 and 3) with a minimum diameter of about 10 mm and a maximum diameter of about 30 mm when inflated, the balloon is substantially spherical when inflated. The balloon is inflated during positioning of the catheter into the pulmonary vein and is typically inflated using fluorescent contrast media. The balloon can be comprised of any of a number of polymers known in the art used in the fabrication of angioplasty balloons (polyesters, polyurethanes, polyamides, polyvinylchlorides, polyethylene terapthylates, etc.).

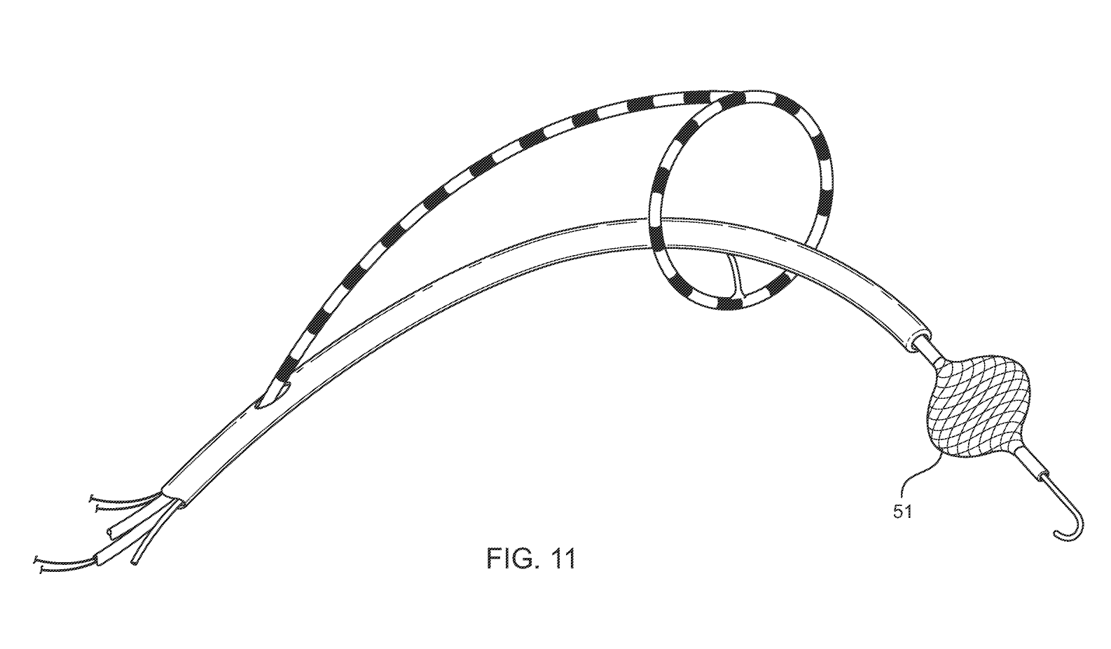

The self-centering anchor member can be a self-expandable basket, 51 (FIG. 11) with a minimum diameter of about 10 mm and a maximum diameter of about 30 mm when expanded. The basket is designed to self-expand after deployment into the pulmonary vein. The basket can be comprised of any of a number of self-expanding metals (for example Nitinol) or shape memory polymers known in the art.

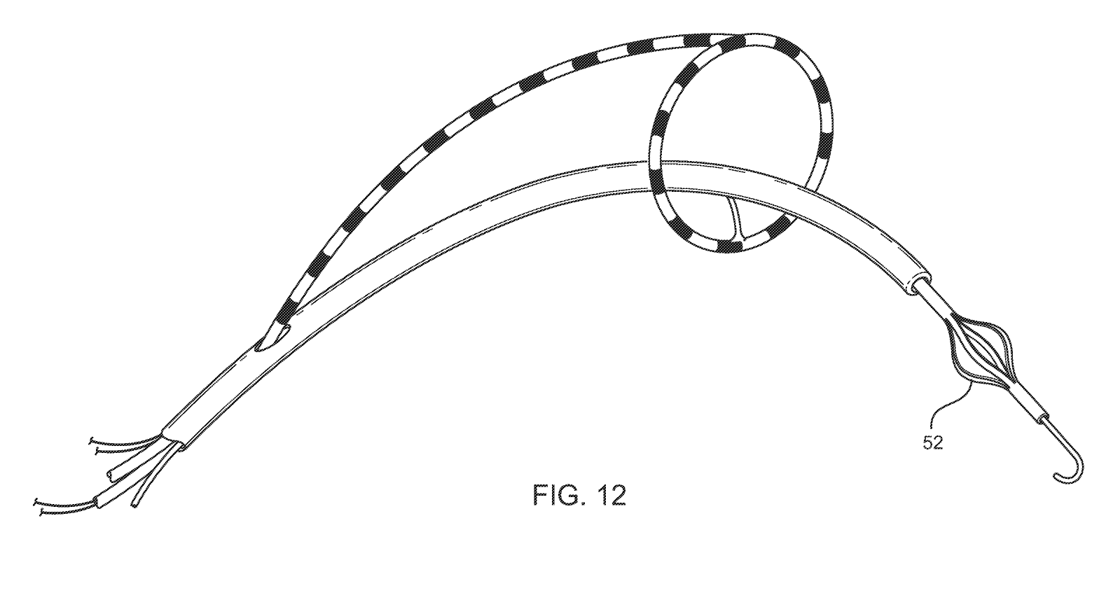

The self-centering anchor member can be a self-expandable malecot, 52 (FIG. 12) with a minimum diameter of about 10 mm and a maximum diameter of about 30 mm when expanded. The malecot is designed to self-expand after deployment into the pulmonary vein. The malecot can be comprised of any of a number of resilient metals (for example a nickel-titanium alloy) or shape memory polymers known in the art.

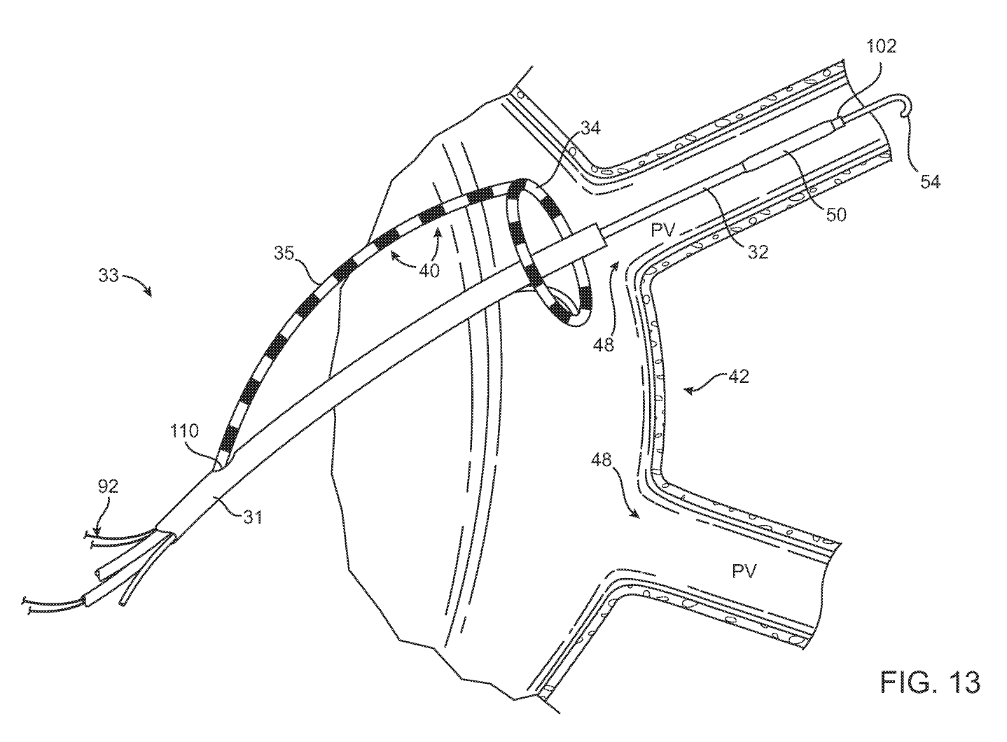

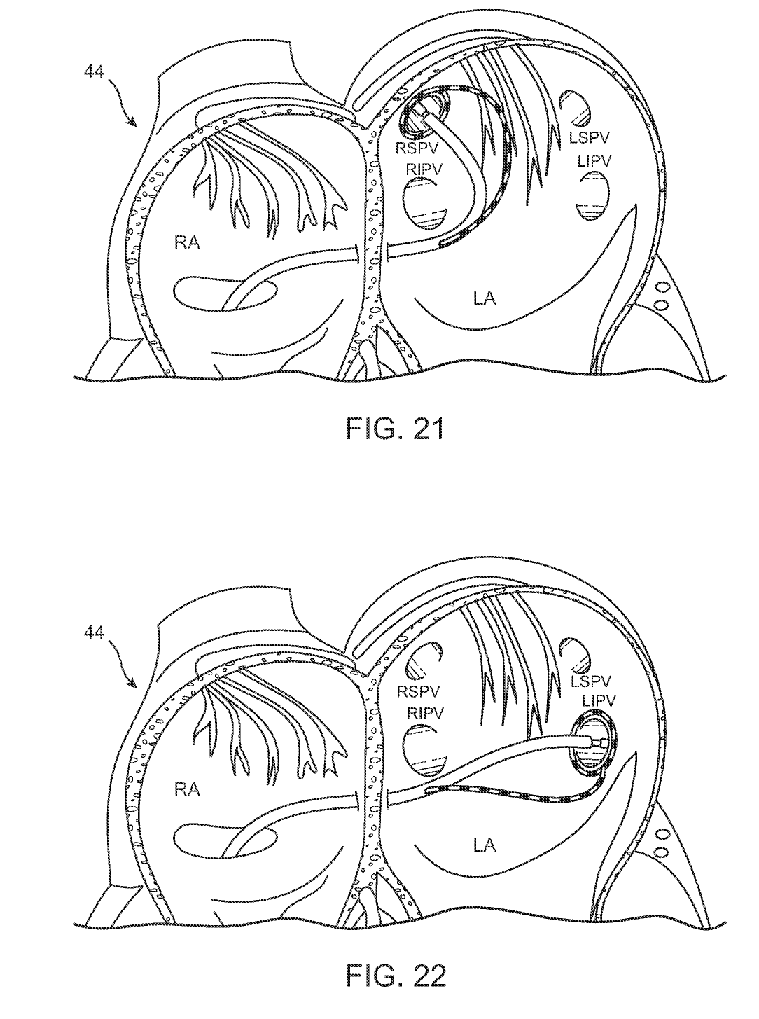

Referring now to FIGS. 13-23, examples of methods for treating atrial fibrillation with the electrophysiological catheter 20 will be described. In some embodiments the method includes ablation in or around the ostium of pulmonary veins, in other embodiments continuous linear lesions are formed on the inner surface of the atrial wall 56, and in yet other embodiments both continuous circumferential and linear lesions can be created.

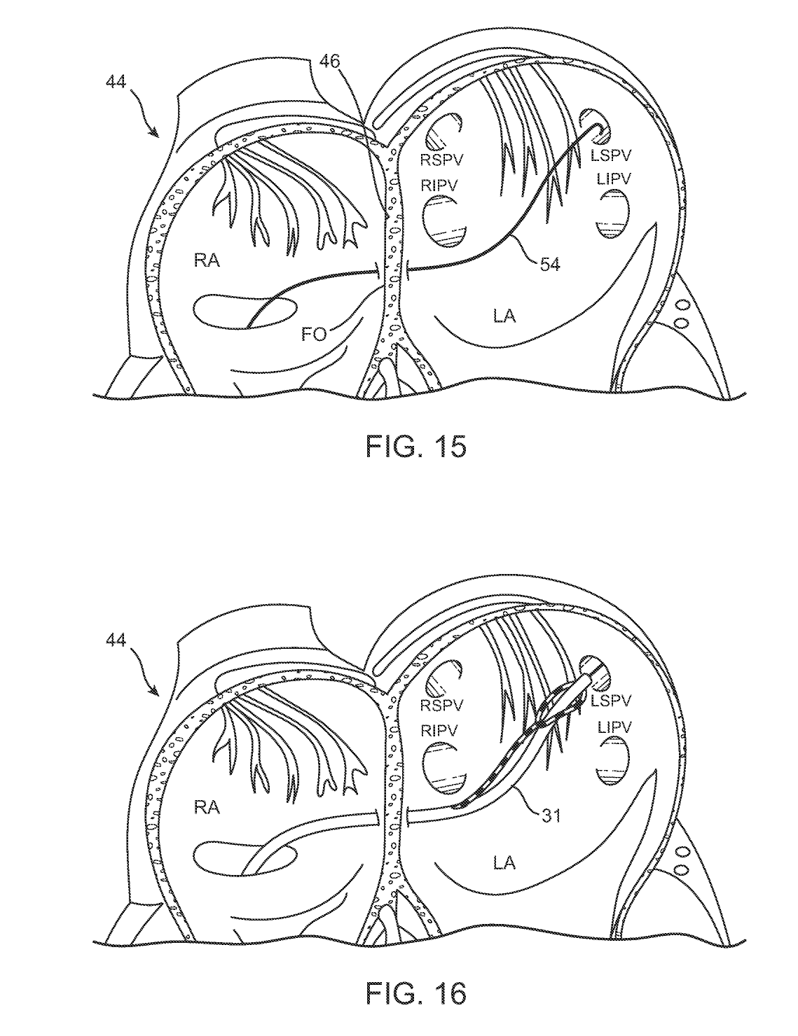

The method includes transseptally accessing the left atrium 42 of the heart 44 of a patient through a puncture in the atrial septum 46 (see FIG. 15), ablating tissue surrounding the four (some patient have less than or more than four pulmonary veins) pulmonary veins 48 (the two superior and the two inferior) in order to isolate them from the rest of the left atrium, and create at least two continuous lines of ablation, one at the roof of the left atrium and one at the mitral isthmus in order to obtain a left atrial segmentation and/or defragmentation.

The methods can be performed using a single transseptal sheath to provide access to a plurality of ablation catheters. One sheath may be used because each of the plurality of ablation catheters may be used to map tissue as well as ablate tissue, thus avoiding the need for both a mapping sheath and an ablation sheath. Transseptally accessing the left atrium with a first catheter is performed by advancing the distal end of the first catheter through a lumen of a trans septal sheath that extends through the puncture of the atrial septal wall.

Depending on operator's choice and on patient's anatomy, either or both isolation of all pulmonary veins and linear ablation of the atrial wall can be obtained with a single catheter or with multiple catheters.

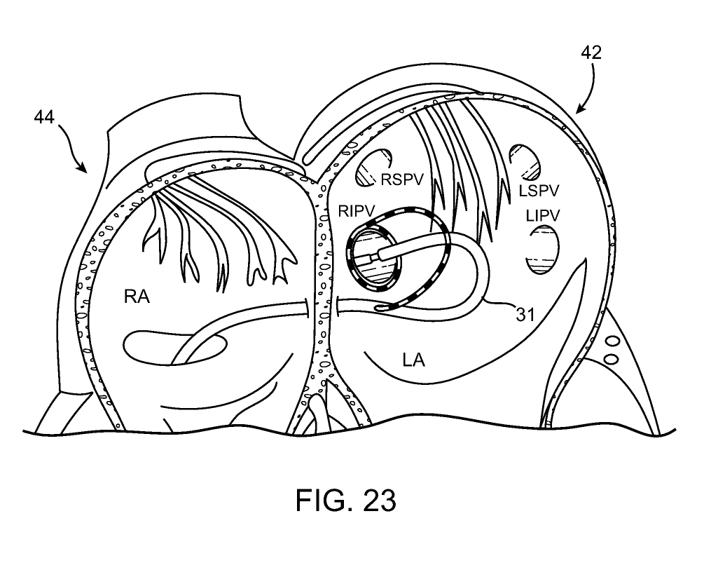

Isolation of both superior pulmonary veins is illustrated in FIGS. 15-20 and 21 and of both inferior pulmonary veins in FIGS. 22 and 23. Prior to performing a pulmonary vein ablation procedure, the method includes withdrawing the transseptal sheath proximally until the distal end thereof is removed from the left atrium 42 of the heart 44. More particularly, withdrawing the transseptal sheath includes retracting the transseptal sheath proximally such that a minimal portion extends into the left atrium 42 or until the distal end thereof is contained in the right atrium of the heart, in order to avoid embolic stroke.

Performing a pulmonary vein ablation procedure further includes sensing electrical signals of the pulmonary vein ostial tissue through one or more electrodes of an electrode array of a catheter. Upon determining that the electrodes 40 on the circumferential structure 34 of this catheter are disposed over an aberrant signal such as an arrhythmogenic focus of the pulmonary vein ostial tissue 48, energy is passed (into the tissue) through the electrodes 40 of the electrode array 36 to ablate a portion thereof.

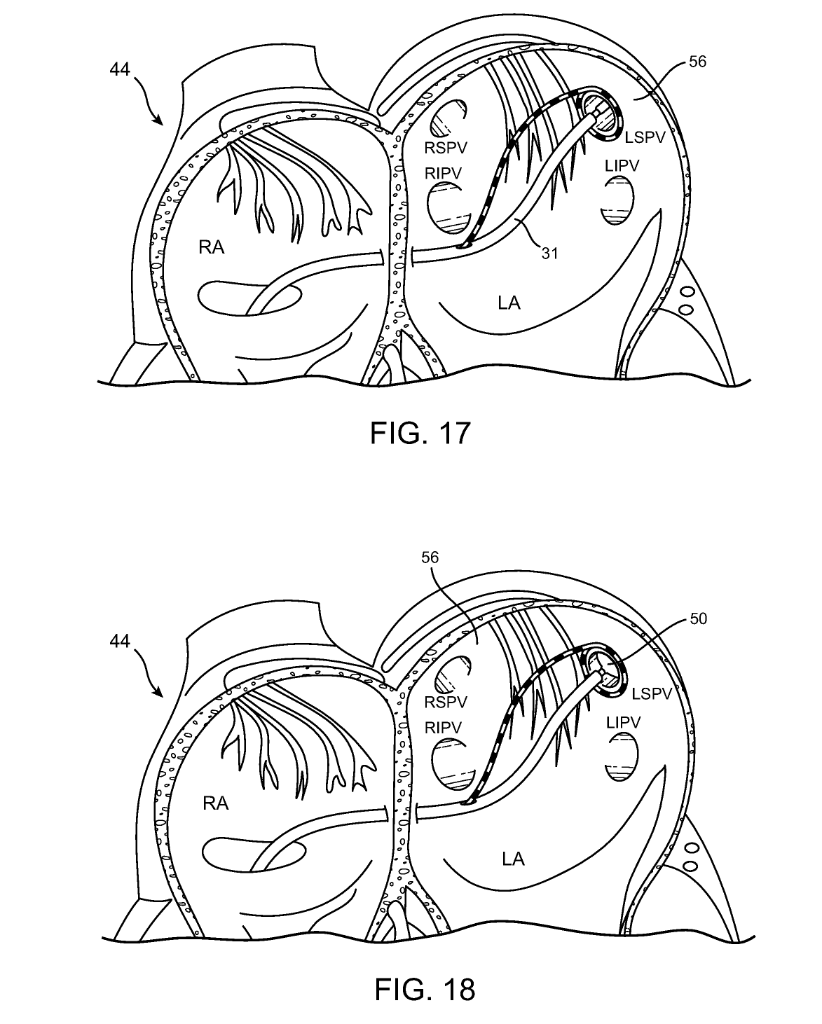

In accordance with another specific configuration, performing a pulmonary vein ablation procedure further includes advancing a first catheter along a guidewire 54 that is selectively inserted into one of the pulmonary veins (FIGS. 15 and 16). If the catheter has a self-centering anchor member 50-52, this member is also inserted over the guidewire 54 into the pulmonary vein (FIG. 17) and inflated or expanded to promote catheter retention at the ostium of the vein (FIG. 18).

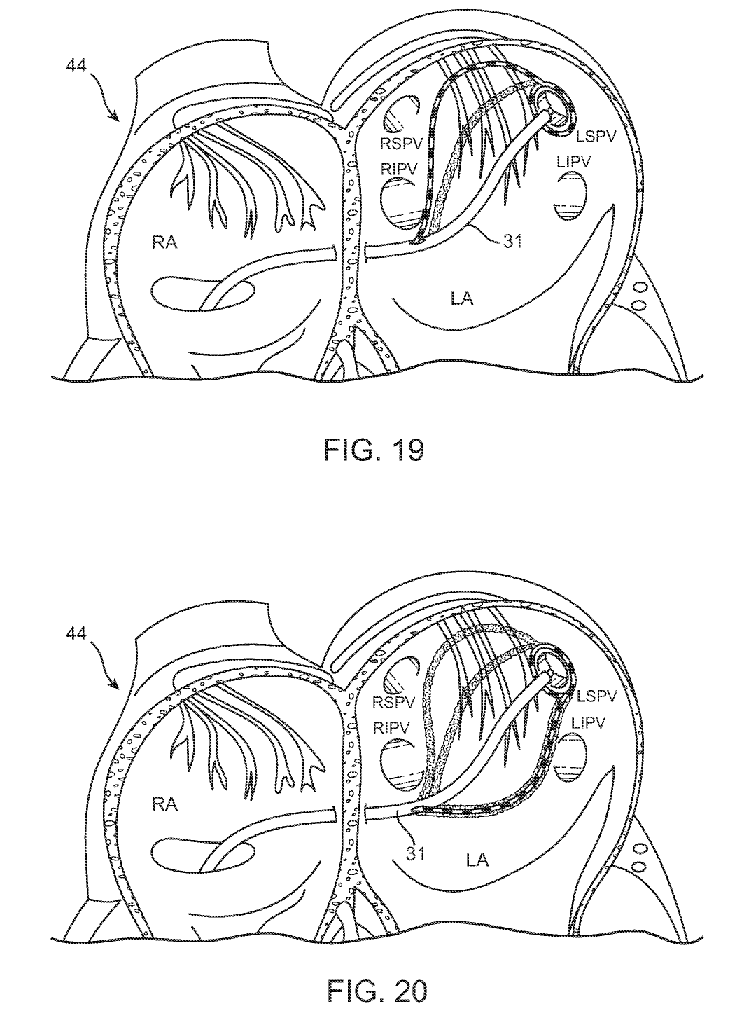

Performing a pulmonary vein ablation procedure further includes selectively moving the circumferential structure 34 of the catheter to other areas of the pulmonary vein ostial tissue 48 surrounding the one or more pulmonary veins, and repeating the sensing of electrical signals and ablating of the pulmonary vein ostial tissue. Contiguous lesions (FIGS. 19 and 20) are created by rotating smoothly the electrode array until 90° about an axis of the first catheter after each repeat cycle. Any of catheters described above may be utilized in these procedures, whereby the lesions isolating the pulmonary veins may be made alone or in combination with linear lesions on the atrial wall, described below, using the same catheter, and avoiding gaps all along the circumferential or linear lesions, by smoothly moving (sliding and/or turning) the catheter.

More specifically, the pulmonary veins ablation procedure is performed in the order of the left superior pulmonary vein first, followed by ablating the ostial tissue surrounding at the left inferior pulmonary vein, which is then followed by the right superior pulmonary vein, and finally the right inferior pulmonary vein.

Various methods of creating linear ablation patterns in the left atrium are illustrated in FIGS. 14, 19 and 20. It should be understood that these patterns and methods are merely exemplary and various other ablation patterns are also possible using the devices and methods of the invention. Performing a left atrial roof 60 linear ablation procedure includes sensing electrical signals of the left atrial roof tissue through one or more electrodes of an electrode array of a catheter. Upon determining that the electrodes of the electrode array of this catheter are disposed over an aberrant signal of the atrial roof tissue, energy is passed through the electrode array of this catheter to ablate a portion thereof.

Similarly, performing a left atrial mitral isthmus 62 ablation procedure includes sensing electrical signals of the mitral isthmus tissue through one or more electrodes of an electrode array of a catheter. Upon determining that the electrodes of the electrode array of this catheter are disposed over an aberrant signal of the mitral isthmus tissue, energy is passed through the electrode array of this catheter to ablate a portion thereof.

If necessary, the same method will be used to create eventually other continuous lines in the left atrium in order to obtain complete left atrial segmentation.

In accordance with another specific configuration, performing a linear ablation procedure further includes advancing a first catheter along a guidewire 54 that is selectively inserted into one of the pulmonary veins (FIG. 15). The catheters described above may be used for this purpose. The circumferential structure 34 of the catheter will be deployed and positioned in contact with the pulmonary vein ostium in order to obtain better stability. If combination linear circumferential ablation catheters are used, the electrode array 36 on the circumferential structure 34 may be used to isolate the pulmonary veins, as described above. Then the linear structure 35 of the same catheter will be deployed in order to get good contact with the left atrial wall 56. The linear electrode array 38 will be moved against the left atrial wall by “push-pull” maneuvers of the whole catheter or using a specific mechanism inside the catheter (control knob 82 controls push/pull wires 92) enabling the deployment of the linear structure 35 in order to obtain a better contact against the left atrial wall. Energy is then delivered to the electrode array 38 to achieve the desired ablation.

Left atrial roof linear lesions will be created by placing the guidewire 54 and the circumferential structure 34 at both left and right superior pulmonary veins (FIGS. 19-20 and 21). If the catheter has a self-centering anchor member 50-52, this member is also inserted over the guidewire 54 into the pulmonary vein (FIGS. 16-18) and inflated or expanded to promote catheter retention at the ostium of the vein.

Mitral isthmus linear lesions will be created by placing the guidewire 54 and the circumferential structure 34 at the left inferior pulmonary vein (FIG. 22). If the catheter has a self-centering anchor member 50-52, this member is also inserted over the guidewire 54 into the pulmonary vein and inflated or expanded to promote catheter retention at the ostium of the vein.

In one configuration, the atrial roof or mitral isthmus ablation procedures are further performed by selectively moving the linear structure 35 of the catheter to other areas of the roof or mitral isthmus tissue, and repeating the sensing electrical signals and ablating of the roof or mitral isthmus tissue. Selectively moving the linear structure 35 of the catheter includes incrementally rotating the electrode array about an axis of the catheter after each repeat cycle. Such incremental rotation of the electrode array about the axis of the catheter is in the range of about 5° to about 15°, this is illustrated in FIGS. 19 and 20.

Other linear lesions (between the right pulmonary veins, between the left pulmonary veins, the floor of the left atrium, the posterior wall, etc.) can be created depending from which pulmonary vein the guidewire 54 and/or self-centering anchor member 50-52 and the circumferential structure 34 of the catheter will be placed. This means that this catheter shouldn't be used without its circumferential structure been first positioned in or around one of the pulmonary veins in order to obtain continuous linear lesions and to avoid left atrial complications. In addition, linear lesions performed along the posterior wall will require special attention and caution in order to avoid esophageal injury.

Using the catheters and methods of the invention, the isolation of the pulmonary veins as well as the linear lesions described above may be accomplished using a single catheter in one intervention. Being equipped with both a circumferential structure 34 configured to encircle the pulmonary vein ostia, as well as a linear structure 35, each optionally with a plurality of electrodes 40 thereon, suitable for circumferential and/or linear ablation of the pulmonary vein ostia and atrial wall respectively, the catheters of the invention allow more precise and stabilized ablation procedures while reducing the time, cost, and potential morbidity associated with multiple-catheter procedures.

While the invention has been described in the context of treating atrial fibrillation, it should be understood that the catheters and methods of the invention may also be used for diagnosis or treatment of other diseases and conditions susceptible to electrophysiological techniques, both within and outside the heart.

In addition, while the catheters have been described as employing radiofrequency energy for ablation purposes, it will be understood that various other energy sources and ablation techniques are possible including ultrasonic, cryoablation, laser ablation, and others. In such cases the catheters of the invention will include the appropriate energy delivery means, ultrasonic transducers, electrodes, optical fibers, cryogenic fluid delivery lumens, or the like as needed for such alternative ablation techniques. However, the basic principles of the invention which enable the creation of both circumferential lesions to isolate the pulmonary veins and linear lesions on the atrial wall using the same catheter will be applicable regardless of the ablation technology utilized.

What is claimed is:

1. An electrophysiological ablation catheter comprising:
an elongated flexible shaft having a distal extremity and a proximal extremity having a connector hub, said distal extremity being adapted for insertion into an atrium of a patient's heart;
a circumferential tissue ablation structure deployably coupled on the distal extremity of the shaft;
a linear tissue ablation structure deployably coupled to the distal extremity of the shaft, wherein the linear ablation structure intersects the circumferential electrode structure at least one point; and
an energy conduction structure extending between the tissue ablation structures and the connector hub;
wherein the circumferential tissue ablation structure is adapted to engage a first venous ostium while the linear tissue ablation structure extends radially from said ostium toward a second venous ostium to produce a substantially continuous circular lesion in or around said first ostium and a substantially continuous linear lesion between said first ostium and second ostium.

2. An electrophysiological ablation catheter as in claim 1, wherein said distal extremity is adapted for insertion into a left atrium, the circumferential electrode is adapted to engage one of the pulmonary veins and the linear electrode structure extends radially toward another pulmonary vein.

3. An electrophysiological ablation catheter as in claim 1, wherein at least one of the circumferential tissue ablation structures and the linear tissue ablation structure includes an array of electrodes spaced to create a substantially continuous lesion when energized in contact with tissue.

4. An electrophysiological ablation catheter as in claim 3, wherein both of the circumferential tissue ablation structures and the linear tissue ablation structure include an array of electrodes spaced to create a substantially continuous lesion when energized in contact with tissue.

5. An electrophysiological ablation catheter as in claim 3, wherein the energy conductor structure comprises a plurality of electrical conductors which connect to individual electrodes so that said electrodes can be selectively activated individually or simultaneously in discreet groups to produce continuous lesions of varying diameter or length.

6. An electrophysiological ablation catheter as in claim 1, wherein the circumferential tissue ablation structure has a diameter from about 20 mm to about 30 mm suitable for the creation of both small and large diameter lesions in or around the venous ostium.

7. An electrophysiological ablation catheter as in claim 1, wherein the linear tissue ablation structure has a length from about 20 mm to about 100 mm suitable for the creation of both short and long linear lesions.

8. An electrophysiological ablation catheter as in claim 1, wherein the catheter includes passages that allow flow of an aqueous solution out of an inner cavity of at least one electrode.

9. An electrophysiological ablation catheter as in claim 1, further comprising a mechanism for deflecting and steering the distal extremity of the shaft, wherein the circumferential support structure can be used as an anchor and placed in or around any of the pulmonary vein ostia within the left atrium or other anatomical structures within the heart.

10. An electrophysiological ablation catheter as in claim 1, further comprising a torquing mechanism for rotating a distal portion of the shaft about a longitudinal axis.

11. An electrophysiological ablation catheter as in claim 1, further comprising a deployable self-centering anchor member to promote catheter retention at a distal end of the distal extremity of the shaft, wherein said anchor member is deployable within the venous ostium as to position the distal extremity axially and radially relative to the ostium.

12. An electrophysiological ablation catheter as in claim 11, wherein the self-centering anchoring member comprises a balloon.

13. An electrophysiological ablation catheter as in claim 12, wherein the inflated balloon diameter can be varied from 10 mm to 30 mm.

14. An electrophysiological ablation catheter as in claim 12, wherein the balloon is substantially spherical in shape when inflated.

15. An electrophysiological ablation catheter as in claim 12, further comprising a balloon support structure that is longitudinally positionable at or from the distal extremity of the shaft independently of the circumferential and linear electrode structures.

16. An electrophysiological ablation catheter as in claim 11, wherein the self-centering anchor member comprises an expandable basket.

17. An electrophysiological ablation catheter as in claim 16, wherein the deployable self-centering anchor member comprises a malecot with a plurality of wings designed to promote catheter retention.

18. An electrophysiological ablation catheter as in claim 1, wherein at least one of the circumferential or linear electrode structures comprise a unitary conductor.

19. An electrophysiological ablation catheter as in claim 18, further comprising an insulative sheath positionable over said at least one of the electrode structures to selectively limit exposure to tissue.

20. An electrophysiological ablation catheter as in claim 3, wherein the array of electrodes are connected via bridges to form a single conductive path.

21. An electrophysiological ablation catheter as in claim 20, wherein the bridges are recessed so as to not contact the tissue upon deployment of the electrode structure.

22. An electrophysiological ablation catheter as in claim 20, wherein the bridges are insulated so as not to contact tissue upon deployment of the electrode structure.

23. A system comprising:
an electrophysiological ablation catheter as in claim 1;
and a power supply coupled to the catheter wherein said power supply is capable of transferring a tissue ablative energy between the power supply and the tissue ablation structures of the catheter.

24. A system as in claim 23, wherein the power supply is configured to deliver at least one of electromagnetic energy, acoustic energy, thermal energy, and mechanical energy.

25. A system as in claim 24, wherein the power supply delivers radiofrequency energy to the ablation structure(s).

26. A system as in claim 23, wherein the power supply is further configured to sense electrical activity of endocardial tissue in contact with at least one of the electrodes.

27. A system as in claim 23, wherein the power supply displays at least one of tissue temperature and tissue impedance.

28. A system as in claim 25, wherein the power supply delivers monopolar radio frequency to the ablation structures.

29. A system as in claim 25, wherein the power supply delivers bipolar radio frequency to the ablation structures.

30. A method for treating atrial fibrillation, said method comprising:
advancing an electrophysiological ablation catheter to an atrium of a patient's heart;
deploying a circumferential ablation structure from the catheter to engage a first venous ostium;
deploying a linear ablation structure array from the catheter so that said linear ablation structure extends radially from the first venous ostium toward a second venous ostium; and
energizing said circumferential and linear ablation structures to produce a substantially continuous circular lesion surrounding the first venous ostium and a substantially continuous linear lesion on the interior surface of the heart from the first venous ostium toward the second venous ostium.

31. A method for treating atrial fibrillation as in claim 30, further comprising sensing through one or more of said ablation structures to determine the presence of aberrant conductive pathways thereby generating an electrical map of inner surfaces of the heart.

32. A method for treating atrial fibrillation as in claim 30, further comprising positioning a anchoring member coupled to a distal end of the catheter in or adjacent to the first venous ostium to radially and longitudinally position the catheter prior to deploying the circumferential and/or linear ablation structures.

33. A method for treating atrial fibrillation as in claim 30, further comprising:
deploying the circumferential ablation structure from the catheter to engage the second venous ostium;
deploying the linear ablation structure from the catheter so that said linear ablation structure extends radially from the second venous ostium toward a third pulmonary venous ostium; and
energizing said circumferential and linear ablation structures to produce a substantially continuous circular lesion surrounding the second venous ostium and a substantially continuous linear lesion on the interior surface of the heart from the second venous ostium toward the third venous ostium.

34. A method for treating atrial fibrillation as in claim 30, further comprising:
deploying the circumferential ablation structure from the catheter to engage the third venous ostium;
deploying the linear ablation structure from the catheter so that said linear ablation structure extends radially from the third venous ostium toward a fourth venous ostium; and
energizing said circumferential and linear ablation structures to produce a substantially continuous circular lesion surrounding the third venous ostium and a substantially continuous linear lesion on the interior surface of the heart from the third venous ostium toward the fourth venous ostium.

35. A method for treating atrial fibrillation as in claim 30, further comprising:
deploying the circumferential ablation structure from the catheter to engage the fourth venous ostium;
deploying the linear ablation structure from the catheter so that said linear ablation structure extends radially from the fourth venous ostium toward the first venous ostium; and
energizing said circumferential and linear ablation structures to produce a substantially continuous circular lesion surrounding the fourth venous ostium and a substantially continuous linear lesion on the interior surface of the heart from the fourth venous ostium toward the first venous ostium.

36. A method for treating atrial fibrillation as in claim 30, wherein energizing comprises delivering at least one of electromagnetic energy, acoustic energy, thermal energy, and mechanical energy to the ablation structures.

37. A method for treating atrial fibrillation as in claim 36, wherein energizing comprises delivering radiofrequency energy to the ablation structures.

38. An electrophysiological ablation catheter comprising:
an elongated flexible shaft having a distal extremity and a proximal extremity having a connector hub, said distal extremity being adapted for insertion into an atrium of a patient's heart;
a deployable self-centering anchor member at a distal end of the distal extremity of the shaft adapted to promote catheter retention within a first venous ostium in order to position the distal extremity axially and radially relative to said first ostium;
a linear tissue ablation structure deployably coupled to the distal extremity of the shaft, wherein the linear ablation structure deploys radially outwardly from the shaft; and
an energy conduction structure extending between the tissue ablation structures and the connector hub;
wherein the linear tissue ablation structure deploys radially from said first ostium toward a second venous ostium to produce a continuous linear lesion therebetween when the anchor member is deployed in the first venous ostium.

39. An electrophysiological ablation catheter as in claim 38, wherein said anchor comprises a circumferential tissue ablation structure which deploys with the anchor to engage the first venous ostium when the anchor is engaged therein.

40. An electrophysiological ablation catheter as in claim 38, wherein the circumferential tissue ablation is adapted to engage the pulmonary vein ostium while the linear electrode structure extends radially toward another pulmonary vein.

41. An electrophysiological ablation catheter as in claim 38, wherein the linear tissue ablation structure includes an array of electrodes spaced to create a substantially continuous lesion when energized in contact with tissue.

42. An electrophysiological ablation catheter as in claim 41, wherein the energy conductor structure comprises a plurality of electrical conductors which connect to individual electrodes so that said electrodes can be selectively activated individually or simultaneously in discreet groups to produce continuous lesions of varying diameter or length.

43. An electrophysiological ablation catheter as in claim 38, wherein the anchor member has a diameter from about 20 mm to about 30 mm suitable for insertion into both small and large diameter venous ostium.

44. An electrophysiological ablation catheter as in claim 38, wherein the linear tissue ablation structure has a length from about 20 mm to about 100 mm suitable for the creation of both short and long linear lesions.

45. An electrophysiological ablation catheter as in claim 38, wherein the self-centering anchoring member comprises a balloon.

46. An electrophysiological ablation catheter as in claim 45, wherein the balloon is substantially spherical in shape when inflated.

47. An electrophysiological ablation catheter as in claim 46, wherein the inflated balloon diameter can be varied from 10 mm to 30 mm.

48. An electrophysiological ablation catheter as in claim 39, wherein the self-centering anchoring member comprises a expandable loop element.

49. An electrophysiological ablation catheter as in claim 48, wherein the expandable loop element is coupled to the linear tissue ablation structure.

50. An electrophysiological ablation catheter as in claim 38, further comprising an anchor structure that is longitudinally positionable at or from the distal extremity of the shaft independently of the linear electrode structure.

51. An electrophysiological ablation catheter as in claim 38, wherein the self-centering anchor member comprises an expandable basket.

52. An electrophysiological ablation catheter as in claim 38, wherein the deployable self-centering anchor member comprises a malecot with a plurality of wings designed to promote catheter retention.

53. A system comprising:
an electrophysiological ablation catheter as in claim 38;
and a power supply coupled to the catheter wherein said power supply is capable of transferring a tissue ablative energy between the power supply and the tissue ablation structures of the catheter.

54. A system as in claim 53, wherein the power supply is configured to deliver at least one of electromagnetic energy, acoustic energy, thermal energy, and mechanical energy.

55. A system as in claim 54, wherein the power supply delivers radiofrequency energy to the ablation structure(s).
